(19) United States
(12) Patent Application Publication
(10) Pub. No.: US 2026/0105866 A1
(43) Pub. Date: Apr. 16, 2026

(54) SYSTEM FOR SIMULATING LAPAROSCOPIC SURGICAL PROCEDURES

(71) Applicant: Makam RAMESH, Bengaluru (IN)

(72) Inventors: Makam RAMESH, Bengaluru (IN); Santhosh S KUMAR, Bengaluru (IN); Tulip ALPHONSO, Bengaluru (IN)

(21) Appl. No.: 19/358,056

(22) Filed: Oct. 14, 2025

(30) Foreign Application Priority Data
Oct. 14, 2024 (IN) 202441077882

(51) Int. Cl. G09B 23/30; A61B 17/00; A61B 18/08; G09B 23/34

(52) U.S. Cl. CPC G09B 23/303; G09B 23/34; A61B 17/00234; A61B 18/082

(57) ABSTRACT

The present disclosure relates to a system for simulating laparoscopic procedures to provide a realistic training environment. The system comprises a simulated abdominal cavity incorporating one or more artificial organs configured to replicate the anatomical form, texture, and response of biological tissues. A laparoscopic hand instrument having a proximal end and a distal end is provided, with the distal end insertable through simulated incisions in the abdominal cavity. The hand instrument includes a thermal dissection unit with a heating element to facilitate controlled dissection of the artificial organs. The system further includes an anastomosis kit having an artificial tubular organ and a circulation unit, configured to simulate anastomosis procedures of vascular and tubular structures. A plurality of interchangeable laparoscopic tools are operable with the hand instrument, to perform the anastomosis procedures on the one or more artificial organs.

### RELATED APPLICATION

The present application claims priority Indian Patent Application No. 202441077882, filed Oct. 14, 2024, the entire contents of which are hereby incorporated by this reference.

### TECHNICAL FIELD

The present subject matter relates, in general, to minimally invasive surgical techniques, and, particularly, to a system for simulating laparoscopic surgical procedures.

### BACKGROUND

Laparoscopic surgery, also referred to as minimally invasive surgery, is a modern surgical technique in which operations may be performed through small incisions (usually 0.5-1.5 cm) as opposed to the larger incisions needed in traditional open surgeries. During the procedure, a laparoscope which is a long, thin telescope transmitting high-intensity light and attached to a high-resolution camera is inserted through a small incision in the abdominal wall of a patient. The camera transmits images of the inside of the abdominal cavity to a video monitor in the operating room, allowing surgeons to perform the surgery by viewing the screen. This technique may offer numerous benefits to patients, including reduced post-operative pain, shorter hospital stays, faster recovery times, and smaller scars. However, laparoscopic procedures require a unique set of skills from the surgeons, as surgeons must operate using long instruments while viewing a two-dimensional video screen.

Training in laparoscopic techniques typically involves a combination of theoretical instruction and practical skill development. Practical training is often facilitated through the use of box trainers, which are mechanical simulators that allow trainees to practice fundamental laparoscopic skills such as hand-eye coordination, instrument navigation, tissue dissection, suturing, and stapling. These simulators may incorporate physical models or virtual reality elements to simulate realistic surgical scenarios. This realistic simulation is important for enabling trainees to rehearse complex laparoscopic procedures in a safe, controlled environment to enhance their proficiency before performing actual surgeries.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1 illustrates a schematic representation of a system for simulating laparoscopic procedures, in accordance with an example implementation of the present subject matter.

FIG. 2 illustrates a thermal dissection unit incorporated as part of the system for simulating laparoscopic procedures, in accordance with an example implementation of the present subject matter;

FIG. 3A illustrates an anastomosis kit of the system for simulating laparoscopic procedures, in accordance with an example implementation of the present subject matter;

FIG. 3B illustrates a circulation unit of the anastomosis kit, in accordance with an example implementation of the present subject matter;

FIG. 4A illustrates one or more organs of a simulated abdominal cavity for performing simulated laparoscopic cholecystectomy procedure, in accordance with an example implementation of the present subject matter;

FIG. 4B-4C illustrate one or more organs from the simulated abdominal cavity for performing simulated laparoscopic cholecystectomy procedure, in accordance with another example implementation of the present subject matter; and

FIGS. 5A-5E illustrate various stages of a simulated laparoscopic cholecystectomy procedure performed within the artificial abdominal cavity, in accordance with an example implementation of the present subject matter.

The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION OF DRAWINGS

In recent years, laparoscopic surgeries have revolutionized minimally invasive surgical procedures by offering patients reduced postoperative pain, shorter hospital stays, and faster recovery times compared to traditional open surgeries. Consequently, the demand for laparoscopic skills training among surgeons and medical professionals has significantly increased.

A primary challenge in laparoscopic procedures involves the surgeon's visualization of abdominal organs on a two-dimensional screen while operating within a three-dimensional environment. This requires the surgeon to maneuver instruments in a direction opposite to the intended movement, a phenomenon known as paradoxical movement, which presents a significant challenge emphasizing the crucial role of psychomotor skills in performing laparoscopic procedures.

Traditional open surgeries primarily rely on a surgeon's dominant right hand, with the left hand assuming a secondary role. However, laparoscopic procedures demand a high degree of ambidexterity, necessitating simultaneous coordination of both hands. Moreover, the fixed approach angle dictated by the initial entry point through the abdominal wall creates a fulcrum effect, causing instruments inside the body to move in the opposite direction of the surgeon's hand movement. This necessitates that the surgeon learns to move the instrument counter-intuitively. Additionally, the magnification of organs through the telescope introduces a notable cognitive hurdle, as increased zoom levels correlate with greater magnification, resulting in a dynamically shifting perspective of organs throughout the surgery.

Furthermore, the use of minute instruments in laparoscopic surgeries presents several challenges. The forces required to manipulate tissues or organs with these instruments differ significantly from those exerted by the entire hand. Consequently, surgeons must undergo extensive training to delicately handle tissues or organs without causing harm, as precise force application is crucial for preventing injuries.

In case of laparoscopic cholecystectomy, a significant challenge is the variability of encountered tissue, influenced by factors such as inflammation and the timing of the surgery. For example, the gallbladder may exhibit diverse pathological changes, including fibrosis and contraction, complicating the procedure. Prolonged stone impacts may further exacerbate such issues, leading to erosion of the gallbladder wall. To navigate these complexities, surgeons must possess a thorough understanding of tissue variations and make informed decisions regarding the most suitable techniques for each condition, which is indispensable for ensuring the safe and successful execution of laparoscopic cholecystectomy.

Existing laparoscopic simulators often come with an expensive price tag, making them inaccessible to many hospitals and training centers. More importantly, these existing simulators frequently struggle to provide a truly lifelike experience of operating on human tissues. They often lack the realistic feeling and feedback that surgeons may need to develop their skills, such as how real tissue responds to touch, how blood flows, or how it reacts during a procedure. Conventionally available laparoscopic simulators often fails to provide mechanisms to assess the quality of laparoscopic procedures, such as suturing or stapling carried out by trainee surgeons. This gap means that even after extensive practice on simulators, users surgeons might still face unexpected challenges when they operate on real patients. Therefore, there is a need for a system that provides a realistic and comprehensive environment for performing simulated laparoscopic procedures.

To this end, the present subject matter provides a system for simulating laparoscopic surgical procedures, enabling a realistic and comprehensive training experience of the laparoscopic procedures by providing an accurate replication of tissue response to the users that may be encountered in live surgery and ensuring that the users develop truly tactile and responsive surgical skills.

In an example, a system for simulating laparoscopic procedures is provided herein. The system comprises a simulated abdominal cavity comprising one or more artificial organs configured to mimic anatomy of a living being. In an example, the system further comprises a laparoscopic hand instrument configured to perform laparoscopic procedures, the laparoscopic hand instrument having a proximal end and a distal end, with the distal end configured to be inserted into the simulated abdominal cavity through one or more incisions provided in the simulated abdominal cavity. In an example, the laparoscopic hand instrument comprises a thermal dissection unit configured to dissect one or more artificial organs, the thermal dissection unit comprises a heating element positioned at a tip of the distal end of the laparoscopic hand instrument. Further, the system comprises an anastomosis kit for simulating anastomosis procedures of vascular and tubular structures associated with the one or more artificial organs. The laparoscopic hand instrument also comprises a plurality of tools configured to perform various actions including anastomosis procedures on the one or more artificial organs.

By providing a simulated abdominal cavity with anatomically accurate artificial organs and a laparoscopic hand instrument equipped with a thermal dissection unit, the present system facilitates realistic replication of laparoscopic surgical procedures in a safe and controlled environment. The arrangement enables users to practice dissection, anastomosis, and other laparoscopic actions with tactile feedback and procedural accuracy comparable to live surgery, while eliminating risks to patients. The inclusion of an anastomosis kit and a plurality of interchangeable tools allows complete training on vascular and tubular reconstructions, thereby improving surgical skills, reducing the learning curve, and enhancing preparedness for actual operative scenarios.

The present subject matter is further described with reference to the accompanying figures. It should be noted that the description and figures merely illustrate the principles of the present subject matter. It is thus understood that various arrangements may be devised that, although not explicitly described or shown herein, encompass the principles of the present subject matter. Moreover, all statements herein reciting principles, aspects, and examples of the present subject matter, as well as specific examples thereof, are intended to encompass equivalents thereof.

Although embodiments for methods and systems for the present subject matter have been described in a language specific to structural features and/or methods, it is to be understood that the present subject matter is not necessarily limited to the specific features or methods described. Rather, the specific features and methods are disclosed as exemplary embodiments for the present subject matter.

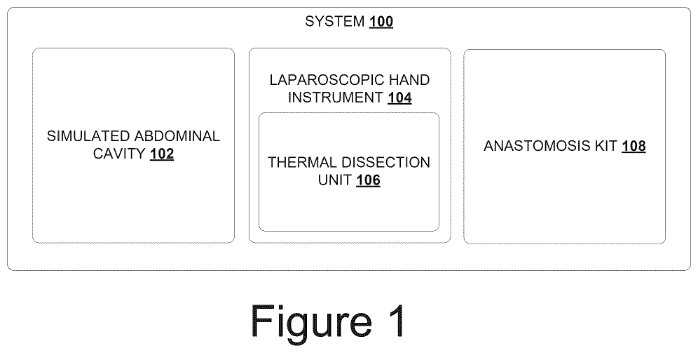

FIG. 1 illustrates a schematic of a system 100 for simulating laparoscopic procedures, in accordance with an example implementation of the present subject matter.

As explained previously, laparoscopic procedures are minimally invasive surgical techniques performed through small incisions using specialized instruments, thereby reducing patient trauma and recovery time compared to open surgery. However, the precision and coordination required for such procedures demand extensive hands-on practice, which is often limited by availability of cadaveric specimens, ethical considerations, and operating room access. Accordingly, a training is required to acquire and refine laparoscopic skills in a controlled, repeatable, and risk-free environment while closely replicating real surgical conditions. In accordance with example embodiments of the present subject matter, the system 100 for simulating laparoscopic procedures as described herein serves as a system for training for performing laparoscopic procedures. The system 100 may be usable by medical students, surgical residents, practicing surgeons, veterinary surgeons, and other healthcare professionals to acquire, refine, and assess minimally invasive surgical skills. In an example, the system 100 may be configured for use in training procedures related to humans as well as animals, thereby enabling both medical and veterinary applications.

As depicted in FIG. 1, the system 100 for simulating laparoscopic procedures comprises a simulated abdominal cavity 102 housing one or more artificial organs configured to replicate the anatomical structure, spatial arrangement, and tactile properties of corresponding organs in a living being. The system 100 further comprises a laparoscopic hand instrument 104 configured to perform laparoscopic procedures.

As will be understood, the laparoscopic hand instrument 104 is a specialized surgical tool used in live laparoscopic procedures. In an example, the laparoscopic hand instrument 104 comprises an elongated shaft extending between a proximal end, configured to be held and manipulated by a user, and a distal end, configured for insertion into the simulated abdominal cavity 102 through one or more incisions. The elongated shaft may be rigid or semi-flexible and is dimensioned to match standard laparoscopic access ports, thereby providing realistic resistance and maneuverability during simulated procedures. The distal end is operatively coupled to a thermal dissection unit 106 (explained subsequently), which includes a heating element located at its tip for simulating thermal cutting, cauterization, or tissue separation. The laparoscopic hand instrument 104 may further include one or more interchangeable tools, such as, but not limited to graspers, scissors, or needle holders, enabling the performance of various laparoscopic actions.

In an example, the system 100 may also provide for simulation of connecting one or more artificial organs or parts thereof using specialized tools and techniques to ensure a secure and functional union. Such connections, particularly in medical or biological applications involving structures, are frequently referred to as anastomoses. To facilitate these training on such intricate connections, in accordance with an example embodiment of the present subject matter, the system includes an anastomosis kit 108 that provides for simulating anastomosis procedures of vascular and tubular structures associated with the one or more artificial organs.

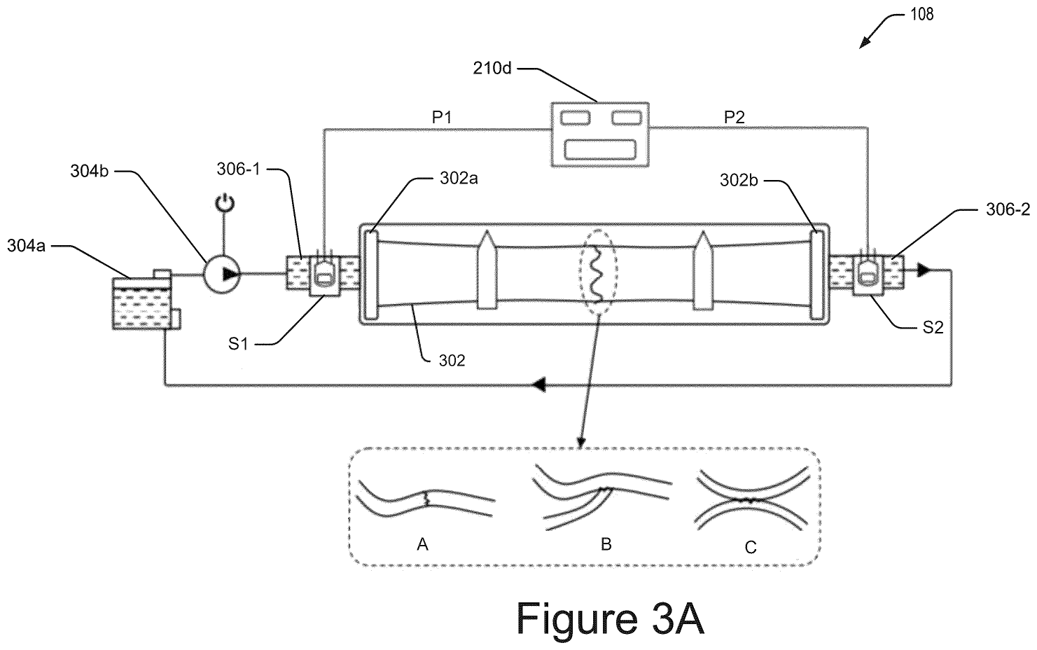

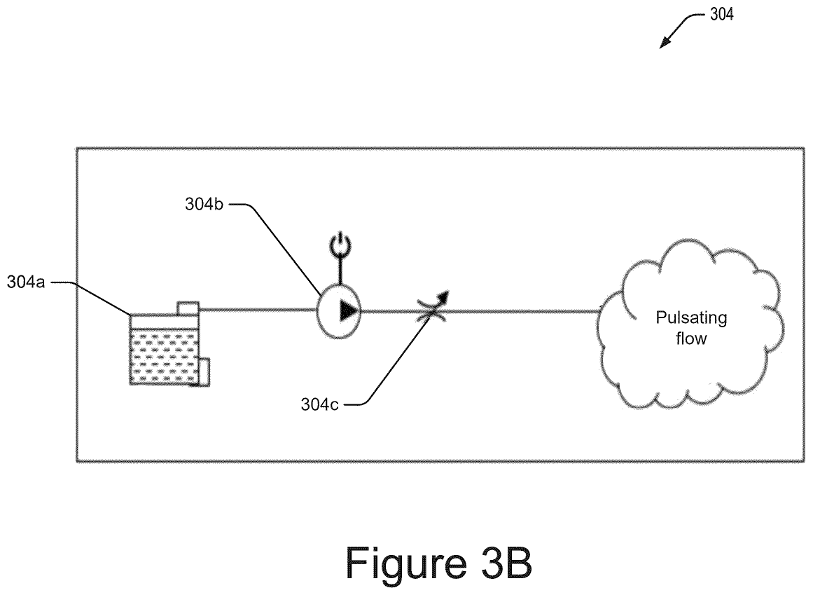

In an example, the anastomosis kit 108 comprises an artificial tubular organ associated with the one or more artificial organs, the artificial tubular organ having a first end and a second end. The artificial tubular organ is configured to represent various biological conduits, including but not limited to arteries, veins, intestines, or other hollow anatomical structures that may require surgical joining or reconnection. The structural and material properties of the artificial tubular organ may be selected to replicate the dimensional, tactile, and suturing characteristics of corresponding biological tissues. The anastomosis kit 108 is illustrated and described in further detail with reference to FIG. 3.

By incorporating the present simulated abdominal cavity having a laparoscopic hand instrument integrated with a thermal dissection unit, the system 100 enables replication of laparoscopic surgical procedures within a controlled, and repeatable training environment. This configuration allows users to perform dissection, anastomosis, and other laparoscopic tasks with realistic tactile response and procedural accuracy closely resembling live surgical conditions, while completely eliminating patient risk. The provision of an anastomosis kit facilitates practice of vascular and tubular reconstructions. Such capability enhances surgical proficiency, shortens the training period, and improves readiness for real-world operative situations.

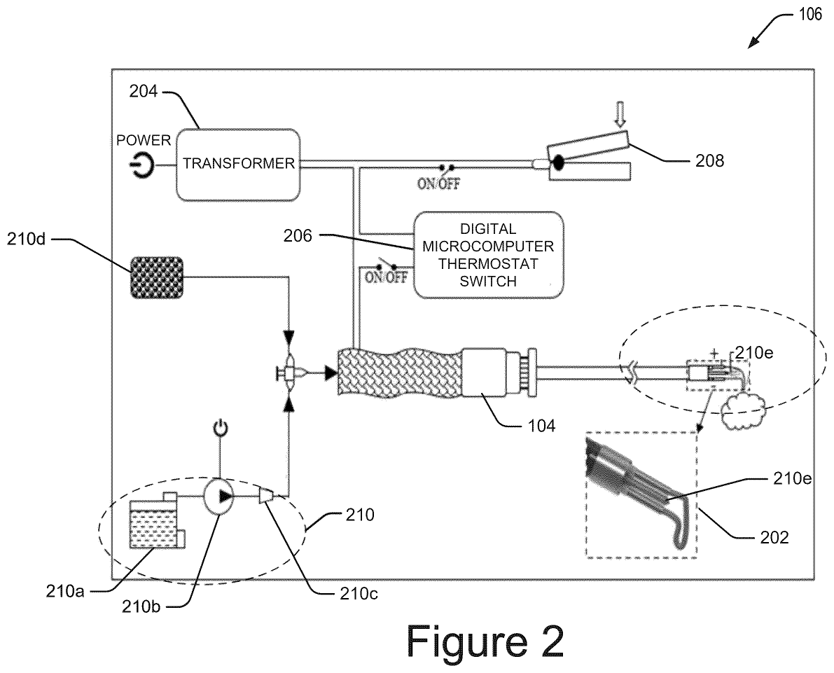

FIG. 2 illustrates a thermal dissection unit 106 incorporated as part of the system 100 for simulating laparoscopic procedures, in accordance with an example implementation of the present subject matter.

As explained previously, the thermal dissection unit 106 is a part of the hand instrument and is configured to facilitate the dissection of one or more artificial organs. The specific composition and structural configuration of the materials forming the artificial organs are described subsequently.

As depicted in FIG. 2, the thermal dissection unit 106 incorporates a set of core components configured to generate and control thermal energy at the distal tip of the laparoscopic hand instrument 104, including a heating element 202, for the purpose of dissecting one or more artificial organs of the simulated abdominal cavity 102. In an example, the thermal dissection unit 106 comprises a transformer unit 204, electrically coupled to the heating element 202 positioned at the distal tip of the laparoscopic hand instrument 104. The transformer unit 204 is configured to step down and regulate the incoming electrical supply to an appropriate voltage and current suitable for the heating element's operational parameters. This regulation ensures safe and efficient conversion of electrical energy into localized heat at the tip, enabling precise dissection.

In one example, the thermal dissection unit 106 includes a microcomputer-controlled thermostat switch 206 configured to regulate the operation of the heating element 202 based on continuous temperature monitoring. The thermostat switch 206 receives input from one or more integrated thermal sensors (not shown) positioned in proximity to the heating element 202, enabling real-time detection of its operating temperature. Based on this feedback, the thermostat switch 206 dynamically modulates the electrical current delivered by the transformer unit 204 to maintain the heating element's temperature within a predefined operational range, the range being determined according to the material properties and thermal response characteristics of the one or more artificial organs being dissected or at a user-defined temperature setpoint. In an example, the microcomputer thermostat switch 206 is configured to turn the transformer unit 204 OFF when the temperature of the heating element 202 reaches the predetermined set value corresponding to the temperature required to dissect one or more artificial organ, and to turn the transformer unit ON when the temperature falls below said set value by a predefined threshold.

In an example, the thermal dissection unit 106 further comprises a foot pedal switch 206 operable to actuate the transformer unit 204 to allow or disrupt flow of electrical current to the heating element. This allows the trainee to maintain both hands on the laparoscopic instrument while performing the simulation training. In an example, the heating element 202 may be a steel wire or a Kanthal wire manufactured using resistive alloy wire (e.g., nichrome or stainless steel) extending through the shaft of the laparoscopic hand instrument 104 and terminating at its distal tip. In an example, the wire may have a stepped diameter, larger along its proximal length for efficient conduction and progressively tapering toward a finer diameter at the distal end. This design increases electrical resistance at the tip, concentrating heat generation precisely where dissection is required.

In operation, the transformer unit 204 may receive an electrical input from a mains supply or dedicated power source. This electrical input may be converted into a regulated low-voltage, high-current output, which is optimized for resistive heating. This electrical output is delivered to the heating element 202. When the user operates the foot pedal switch 208, the transformer unit 204 is activated, and electrical current flows through the heating element 202. The microcomputer thermostat switch 206 continuously monitors the temperature via embedded sensors, adjusting current delivery in real time to maintain the target setpoint. As the tip of the heating element 202 reaches the desired temperature, the concentrated heat is applied directly to the one or more artificial organ surfaces within the simulated abdominal cavity, enabling the users to cut or dissect synthetic tissue with tactile and thermal feedback closely resembling live laparoscopic surgery.

In an example, the cooling unit 210 may comprise a fluid-based cooling mechanism in which the cooling medium is a liquid, such as water, Glycol-based coolants, or any other coolant. The cooling unit 210 in this embodiment may include a fluid tank 210a configured to store the liquid coolant, a pump 210b implemented as a water pump or liquid circulation pump, and a flow controller 210c configured to regulate flow rate and pressure. In an example, the pump 210b delivers the coolant from the tank 210a through the internal channels of the laparoscopic hand instrument 104 to one or more nozzles 210e located at the distal end of the instrument. The liquid coolant is expelled from the nozzles 210e directly onto or around the heating element 202, thereby dissipating heat and preventing thermal damage to the artificial tissues. In an example, the coolant stored in the fluid tank 210a may be pre-cooled by a refrigerating element before circulation, thereby enhancing cooling efficiency and reducing cooldown time between successive simulated dissections.

In an alternate embodiment, the cooling unit 210 may be operated with air as the cooling medium. In such a case, a pump 210d, implemented as an air pump, a mini-compressor, or a vacuum pump depending on the required training scenario, may be used to deliver a continuous or pulsed stream of pressurized air through the internal channels of the laparoscopic hand instrument 104 to the nozzles 210e, thereby achieving rapid thermal reduction of the heating element 202. Conversely, when configured as a vacuum pump, the cooling unit 210 may evacuate heated air or vaporized by-products from the vicinity of the heating element 202, thereby maintaining a clear operative field and simulating suction commonly required during laparoscopic procedures, while also improving cooling efficiency and reducing cooldown times between simulated dissection tasks.

In another example, the cooling unit 210 further comprises one or more external fans (not shown) positioned near the distal end of the laparoscopic hand instrument 104. In an example, these fans may be configured to operate independently or in conjunction with the liquid-or air-based cooling systems, thereby providing a hybrid cooling approach. When activated, the fans can direct high-velocity airflow over and around the heating element 202, accelerating heat dissipation through forced convection. In some examples, the fan speed may be dynamically controlled by the microcomputer thermostat switch 206 in response to real-time temperature readings from thermal sensors positioned at or near the heating element 202.

By integrating the thermal dissection unit 106 with the cooling unit 210, the system 100 delivers realistic simulation environment. The heating element 202 replicates precise cutting and coagulation effects, while the cooling mechanisms prevent overheating, protect artificial organs, and extend component life. This combination enhances procedural accuracy, supports varied surgical scenarios, and reduces the learning curve for users.

FIG. 3A illustrates an anastomosis kit 108 of the system 100, and FIG. 3B illustrates a circulation unit 304 as a part of the anastomosis kit 300, in accordance with an example implementation of the present subject matter.

For clarity both FIGS. 3A and 3B are explained together, as the anastomosis kit 108 and the circulation unit 304 operate in conjunction within the training environment.

As depicted, the anastomosis kit 108 may comprise an artificial tubular organ 302 associated with the one or more artificial organs having a first end 302a and a second end 302b. The artificial tubular organ 302 may be designed to accurately replicate the anatomical structure, elasticity, and lumen diameter of various biological conduits, such as, but not limited to, arteries, veins, ureters, intestines, trachea, or other hollow structures that require surgical reconnection. In an example, the anastomosis kit 108 further includes a circulation unit 304 (as illustrated in FIG. 3B) fluidly coupled to the artificial tubular organ 302 and configured to establish and maintain a controlled flow of fluid from the first end 302a to the second end 302b.

In an example, the circulation unit 304 may include a fluid storage tank 304a configured to contain a fluid medium that may be synthetic blood analogs, saline solution, colored water, or any other liquid capable of visually and physically simulating biological fluids. The tank 304a may be connected to a pulsating pump 304b configured to draw the fluid from the tank 304a and deliver it into the artificial tubular organ 302. The pulsating pump 304b may be operable at variable flow rates and pulsation frequencies to replicate physiological hemodynamic conditions corresponding to different biological vessels, such as high-pressure arterial flow or low-pressure venous return. In an example, a flow control unit 304c may be coupled in-line with the delivery system, along with one or more pipes or flexible conduits, to regulate and direct the movement of the fluid. The system may be configured to generate a pulsating flow, thereby enabling a more realistic simulation of in vivo circulation patterns during training procedures.

In an example, the present anastomosis kit 108 may also allow users to perform different types of anastomosis configurations, as shown in FIG. 3A: an end-to-end anastomosis A, where one tubular organ is sutured directly to another tubular organ; an end-to-side anastomosis B, where one end of the tubular organ is connected to a side opening of another tubular organ; and a side-to-side anastomosis C, where the side walls of two tubular organs are joined along a longitudinal incision to create a parallel fluid pathway. This allows users to practice multiple anastomotic techniques under varied flow conditions, improving adaptability and proficiency in diverse surgical scenarios. In an example, the anastomosis kit 108 may be a portable kit.

In an example, to evaluate or assess the quality of these anastomosis procedures, the circulation unit 304 further comprises a first pressure sensor S1 positioned at the inlet side 306-1 of the first end 302a and configured to measure the inlet flow pressure P1 of the circulating fluid. Similarly, a second pressure sensor S2 is positioned at the outlet side 306-2 of the second end 302b and configured to measure the outlet flow pressure P2 of the fluid. In an example, the readings from the pressure sensors S1 and S2 may be used to monitor flow resistance, detect simulated occlusions or leaks, and provide real-time feedback to the user during various types of anastomosis procedures.

In an example, the inlet flow rate S1 measured by the first pressure sensor P1 and the outlet flow rate S2 measured by the second pressure sensor P2 may be compared both before and after a suturing or stapling process is performed on the artificial tubular organ 302. This comparison provides a direct and objective method for assessing the quality of the suturing or stapling process. For instance, a significant drop in the outlet flow rate S2 compared to the inlet flow rate S1, or a noticeable pressure differential across the anastomotic site, may indicate improper suturing, such as a constricted lumen (stenosis) or an incomplete seal. Similarly, detection of fluid leakage at the sutured or stapled junction can point to a defect in the connection's integrity. In an example, the system 100 may also assess parameters such as leak-proof performance, simulated blood loss, and burst pressure of the connection, with these metrics displayed in real time on a display unit 310. This enables continuous monitoring of fluid loss, internal pressure variations, evaluation of tightness of knots to check any leakages, and overall connection durability during the procedure. The real-time feedback mechanism thus allows immediate evaluation of the trainee's performance, ensuring that the sutured or stapled connection in the artificial tubular organ meets clinical standards of strength and sealing efficiency.

In an example, the anastomosis kit 108 also comprises one or more strain gauges (not shown), positioned on the artificial tubular organ 302, configured to measure a rate of tensile strain induced in the artificial tubular organ 302 during the suturing process. These strain gauges may be specifically designed to monitor and measure the rate of tensile strain, that is, the amount of stretching or elongation that occurs in the artificial tubular organ 302 during the suturing process. This allows for real-time feedback on the stress being applied to the artificial organ 302, which can be critical for assessing the quality of the sutures in the simulated anastomosis procedure.

By incorporating the anastomosis kit 108 into the system 100, users are able to develop and refine critical surgical skills such as vessel handling, precision cutting, suturing, leak testing, and flow control in a safe, controlled, and repeatable environment. The kit enables deliberate practice of high-risk procedural steps without the pressure or consequences of a live clinical setting, thereby enhancing confidence and procedural proficiency before operating on actual patients. It also facilitates objective competency assessment by mentors or training institutions, ensuring that skill acquisition meets defined clinical standards. Owing to its portable and modular design, the kit can be deployed in surgical workshops, space-limited institutions, or even for at-home practice, making advanced laparoscopic training accessible and scalable.

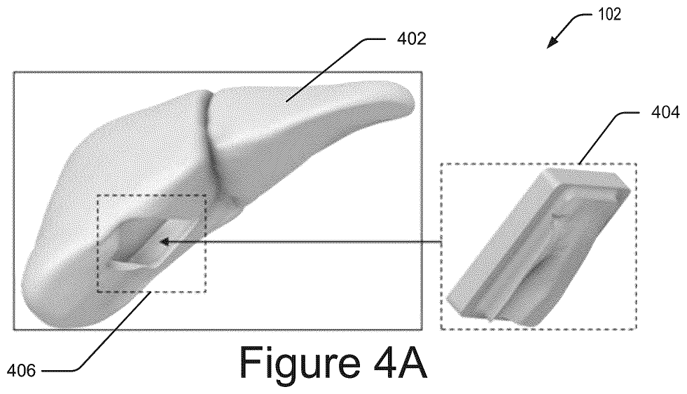

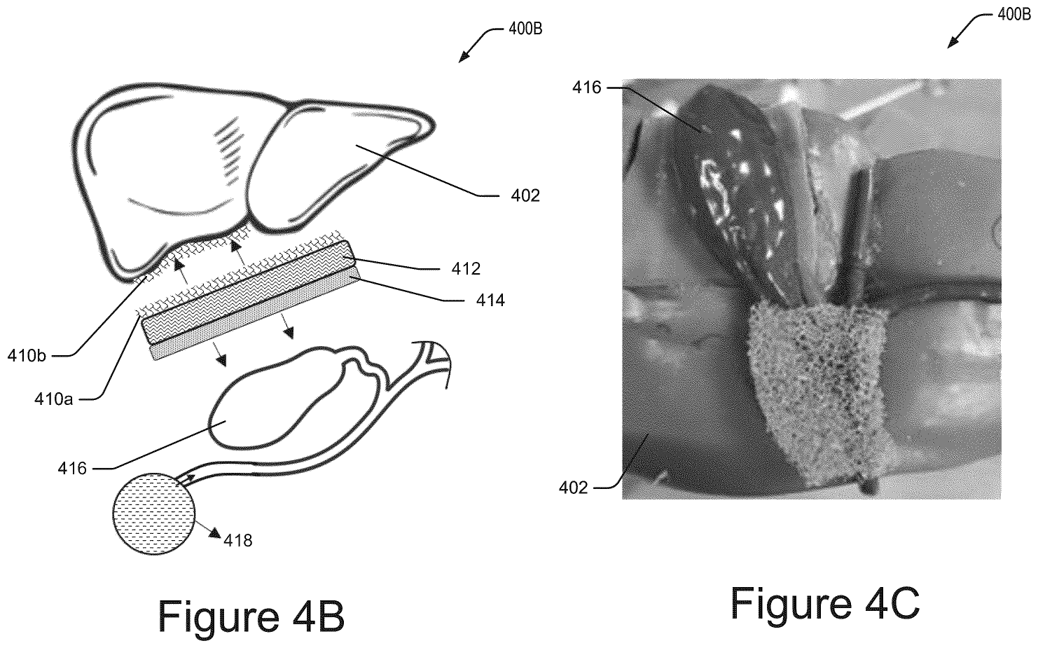

FIG. 4A illustrates one or more organs from the simulated abdominal cavity 102, in accordance with an example implementation of the present subject matter.

As explained previously, the one or more artificial organs of the simulated abdominal cavity 102 are fabricated to accurately replicate the anatomical structure, tactile response, and surface texture of corresponding human tissues and organs. The construction and material composition of these artificial organs are selected to mimic the resistance, elasticity, and feedback encountered during live laparoscopic manipulation, thereby enabling realistic surgical simulation. The simulated abdominal cavity 102 is designed to facilitate training for a variety of laparoscopic procedures, including but not limited to laparoscopic cholecystectomy, a minimally invasive surgical technique for the removal of an infected or diseased gallbladder. Such replication allows surgeons, users, and other medical professionals to practice and refine their procedural skills, instrument handling, and intraoperative decision-making in a safe, controlled, and repeatable environment without posing any risk to actual patients.

As depicted in FIG. 4A, the simulated abdominal cavity 102 illustrates an artificial liver 402 configured to accommodate a replaceable gallbladder assembly (not shown) for laparoscopic cholecystectomy training. The gallbladder assembly is mounted to the artificial liver 402 through a mechanical locking mechanism designed to provide both secure attachment during the procedure and ease of replacement thereafter. In an example, the locking mechanism comprises one or more Velcro® straps (not shown in FIG. 4A) positioned to wrap around designated anchoring regions of the artificial liver 402, thereby securing the gallbladder assembly in a stable operative position. In an example, the gallbladder assembly may be supported on a silicone mounting pad 404, which provides a realistic tissue-like interface for dissection and also serves as an intermediate support surface between the gallbladder and the artificial liver 402. The silicone pad 404, along with the gallbladder assembly, is configured to be inserted into and retained within a slot 406 formed in the artificial liver 402. This slot 406 is dimensioned and shaped to receive the assembly in a manner that prevents undesired movement during simulated surgical manipulation, while enabling quick removal and replacement of the assembly for repeated practice sessions. The arrangement allows users to perform a complete simulated cholecystectomy, detach the used gallbladder model after the procedure, and insert a new gallbladder assembly without the need for specialized tools, thereby supporting high-frequency, repeatable training in both individual and group instructional settings.

In an example implementation, the artificial gallbladder may be fabricated by molding silicone material to achieve a texture and compliance closely resembling that of a biological gallbladder. The fabrication process may be performed using a negative replica mold of the gallbladder's anatomical shape, wherein silicone layers are applied in a sequential layer-by-layer deposition technique to build the required wall thickness and structural integrity. Each layer may be allowed to cure before application of the next, ensuring uniform strength and flexibility across the structure. The completed gallbladder model may be dimensioned to maintain accurate anatomical proportions for realistic surgical simulation.

The artificial liver 402 may be produced by first creating a liver mold using three-dimensional (3D) printing technology based on anatomical reference data. A silicone formulation, prepared in a predetermined mixing ratio of 1:1 by weight of base and curing agent, may be poured into the mold and allowed to cure at room temperature until the desired firmness and elasticity are achieved. To enable attachment of the replaceable gallbladder assembly, a Velcro strip may be fixed at a designated location on the liver surface. This attachment may be reinforced using a mesh substrate and a suturing technique, thereby enhancing mechanical stability and ensuring that the gallbladder assembly remains securely positioned during repeated training cycles.

In another example, the artificial liver 402, along with other artificial organs positioned within the simulated abdominal cavity 102, may be fabricated from silicone-based materials selected to replicate the mechanical properties, elasticity, and tactile response of corresponding biological tissues and may be used as a training tool to simulate laparoscopic procedures. The one or more artificial organs may be formed by mixing Part A and Part B silicone components in a 1:1 volumetric ratio. Part A and Part B silicone refer to the two components of a two-part silicone system, commonly used in liquid silicone rubber (LSR), medical silicones, and addition-cure silicone adhesives. In an example, Part A silicone typically comprises a catalyst and a base polymer, such as, but not limited to, polydimethylsiloxane (PDMS), which gives the silicone its main structure and properties. In an example, Part B may comprise a cross-linker or curing agent, which may react with the catalyst in Part A to form the solid silicone rubber. The silicone may be used for providing durability, flexibility, and similarity to the real organs. In an example, the one or more organs may be casted using a 3D-printed molds for providing precise shape and details. The silicon-based one or more organs, such as a gallbladder, liver and surrounding tissues may be provided for providing a true-to-life simulation of the surgery and may help surgeons and medical professionals practice and refine their skills in performing various laparoscopic surgeries, such as, but not limited to cholecystectomy, which is a minimally invasive surgery to remove the infected gallbladder.

In an example, each of the one or more artificial organs may comprise an artificial tissue, designed to mimic the properties of biological tissue. This artificial tissue component comprises a specific composition to achieve realistic tactile and mechanical characteristics. In an example, the artificial tissue component may comprise a composition of paraffin wax in a range of 70-80 % by volume, polyester synthetic cotton microfibers in a range of 15-20 % by volume, and petroleum jelly in a range of 5-10 % by volume. In an example, silicone glue may also be used for sealing the gaps between the one or more synthetic organs to mimic anatomy of the living being.

In an example implementation, at least a portion of the one or more artificial organs may incorporate an interposed layer of biomimetic material positioned between simulated tissue layers of adjacent artificial organs to emulate the connective tissue planes found in vivo. The biomimetic material may be formulated to replicate the thermal, mechanical, and dielectric properties of natural connective or fatty tissue so as to respond in a realistic manner to surgical energy modalities such as radiofrequency energy, thermal energy, ultrasonic energy, or combinations thereof. Suitable materials for the interposed layer may include, but are not limited to, silicone elastomers of varying durometers, thermoplastic elastomers, polyurethane gels, gelatin-infused meshes, or composite layers comprising woven or non-woven polymer fibers impregnated with water-retaining media. In certain implementations, the interposed layer may comprise plant-derived live cellular tissue, such as freshly harvested vegetable or fruit tissue, selected for its mechanical properties, water content, and ability to mimic connective tissue planes. The thickness, density, and moisture content of the interposed layer may be selectively varied to simulate different surgical difficulty levels, ranging from easily separable planes to dense fibrotic adhesions, thereby providing an adaptable training environment for a variety of surgical skill levels.

By providing the present simulated abdominal cavity offers a highly realistic feel and texture that closely mimics human tissues and organs while maintaining exceptional durability for repeated use. Its anatomically accurate design, including detailed structures such as the gallbladder and surrounding tissues, provides users with a lifelike surgical environment. The optional integration of pulsatile flow enhances the simulation by replicating physiological blood circulation, enabling realistic visualization of intraoperative conditions. Furthermore, the model supports comprehensive skill development by allowing practice in suturing, tissue dissection, manipulation, and handling, thereby preparing users for real surgical scenarios with improved precision and confidence.

FIG. 4B and FIG. 4C illustrate one or more organs from the simulated abdominal cavity 102 for performing simulated laparoscopic cholecystectomy procedure, in accordance with an example implementation of the present subject matter.

As depicted in FIGS. 4B and 4C, the artificial liver 402 is configured to receive a replaceable gallbladder assembly 416 through a modular attachment system designed for repeated installation and removal during training. In an example, one side of a Velcro strip 410a is permanently affixed to a designated attachment region on the surface of the artificial liver 402. The replaceable gallbladder assembly 416 comprises a gallbladder-shaped foam body 412, which may be formed from a blender foam material for simulating standard surgical dissection conditions, or alternatively from a Scotch Brite™ scrubber material to provide increased mechanical resistance for advanced or difficult dissection scenarios. In an example, the modular attachment technique demonstrated for the gallbladder assembly may be extended to other artificial organ systems within the simulated abdominal cavity. This approach enables interchangeable anatomical components to be affixed using standardized interfaces, such as Velcro, magnetic couplings, or snap-fit mechanisms, allowing for repeated installation and removal.

In an example, the foam body 412 is cut to match the top-view profile of a biological gallbladder, thereby ensuring anatomically accurate positioning during attachment. In an example, the foam body 412 is configured as a layered structure, wherein one side is coated with a thin layer of silicone 414 to replicate the smooth, elastic texture of gallbladder tissue, while the opposite side is fitted with a second Velcro strip 410b. The silicone-coated side of the foam body 412 is bonded to the gallbladder 416 using silicone adhesive, ensuring a secure yet realistic attachment interface for dissection training. The Velcro-equipped side of the foam body 412 is operatively coupled to the liver-side Velcro 410a, thereby forming a Velcro “sandwich” connection that allows the gallbladder assembly 416 to be locked into place or detached as required.

In an example, the gallbladder 416 also includes an artificial membrane, such as a balloon 418, filled with a simulated biological fluid. The simulated fluid may be selected from a group consisting of synthetic blood analogs, colored water, or saline solutions with added viscosity modifiers. The artificial membrane 418 may further be coupled to one or more artificial arteries configured to maintain the internal fluid at a constant positive pressure, such that any incision or perforation to the gallbladder 416 during a training procedure results in a controlled fluid leak, thereby replicating intraoperative injury conditions.

The replaceable gallbladder assembly 416 is configured as a modular unit, enabling quick removal from the artificial liver 402 after completion of a simulated surgical procedure. This modular configuration facilitates rapid replacement with a fresh gallbladder assembly for subsequent training sessions. The Velcro-based locking system ensures a secure fit during operation while allowing for straightforward detachment without damage to the liver or gallbladder components. This design allows users to practice multiple cycles of gallbladder removal, attachment, and leak testing under varying difficulty levels, thereby improving their precision and confidence in performing laparoscopic cholecystectomy.

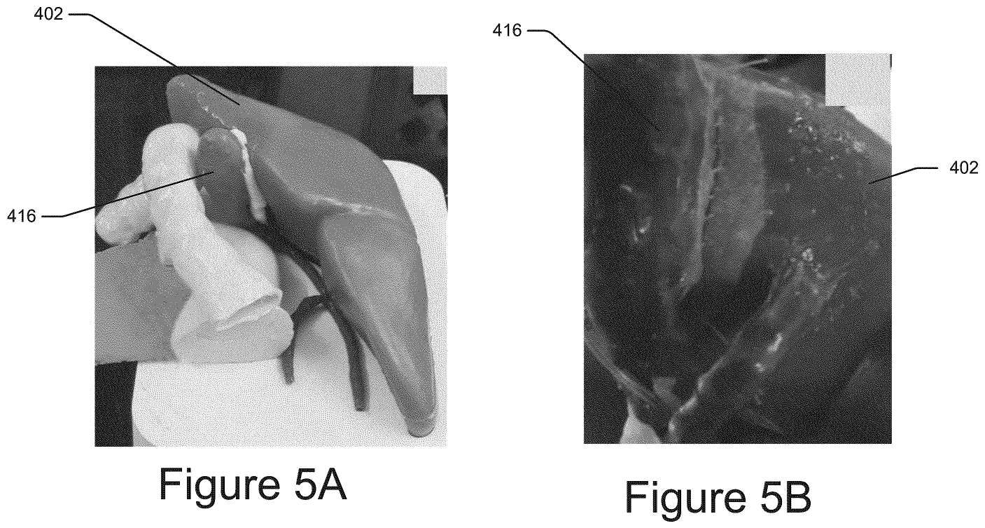

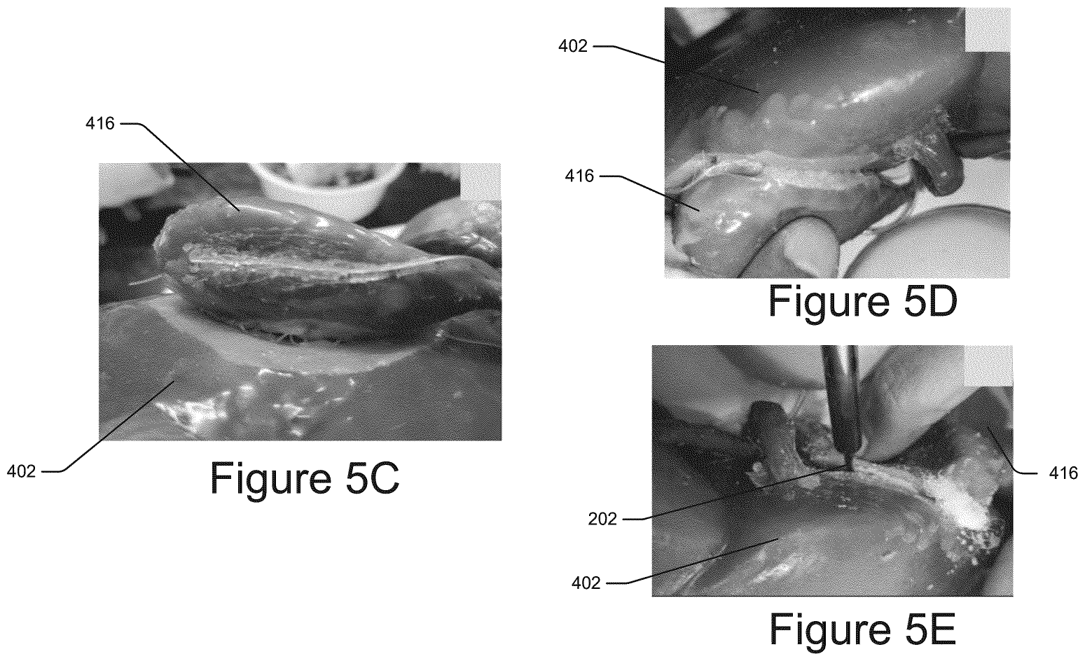

FIGS. 5A-5E illustrate various stages of a simulated laparoscopic cholecystectomy procedure performed within the artificial abdominal cavity 102, in accordance with an example implementation of the present subject matter. These figures depict the steps undertaken by a user to perform the simulated procedure. FIGS. 5A to 5E are described in conjunction to illustrate the stages of the simulated laparoscopic cholecystectomy procedure.

As depicted, FIG. 5A shows the overall laparoscopic cholecystectomy training model comprising the artificial liver 402, the artificial gallbladder 416, and other associated artificial organs arranged to mimic the anatomical layout of the human abdominal cavity 102.

FIG. 5B illustrates the mesh arrangement and suturing technique employed to secure the gallbladder 416 to the liver 402, enabling users to practice fixation and tissue-handling skills.

FIGS. 5C and 5D depict the artificial connective tissue provided between the liver 402 and the gallbladder 416, designed to simulate the fibrous and membranous layers encountered in a real surgical procedure.

FIG. 5E illustrates the use of heating element 2020 of the thermal dissection unit 106 to perform dissection during the laparoscopic cholecystectomy procedure, enabling the users to practice energy-based tissue separation while receiving real-time feedback on technique and precision.

Although the subject matter has been described in considerable detail with reference to certain examples and implementations thereof, other implementations are possible. As such, the present disclosure should not be limited to the description of the preferred examples and implementations contained therein.

I/We claim:

1. A system for simulating laparoscopic procedures, the system comprising:
a simulated abdominal cavity comprising one or more artificial organs configured to mimic anatomy of a living being;
a laparoscopic hand instrument configured to perform laparoscopic procedures on one or more artificial organs, the laparoscopic hand instrument having a proximal end and a distal end, wherein the distal end is to be inserted into the simulated abdominal cavity through one or more incisions provided in the simulated abdominal cavity, wherein the laparoscopic hand instrument comprises:
a thermal dissection unit configured to dissect one or more of the artificial organs, the thermal dissection unit comprises a heating element positioned at a tip of the distal end of the laparoscopic hand instrument; and
an anastomosis kit for simulating anastomosis procedures of vascular and tubular structures associated with the one or more artificial organs,
wherein the laparoscopic hand instrument comprises a plurality of tools configured to perform the anastomosis procedures on the one or more artificial organs.

2. The system as claimed in claim 1, wherein the thermal dissecting unit further comprises:
a transformer unit electrically coupled to the heating element, and configured to supply electric current to the heating element;
a microcomputer thermostat switch electrically coupled to the heating element to regulate the temperature of the heating element by controlling the electric current supplied by the transformer unit; and
a foot peddle switch operable to actuate the transformer unit to allow or disrupt flow of electric current to the heating element.

3. The system as claimed in claim 2, wherein the microcomputer thermostat switch is configured to turn the transformer unit OFF when the temperature of the heating element reaches a predetermined set value corresponding to the temperature required to dissect one or more artificial organ, and to turn the transformer unit ON when the temperature falls below said set value by a predefined threshold.

4. The system as claimed in claim 1, wherein the heating element comprises a wire to concentrate heat at the tip for dissecting the one or more artificial organ,
wherein the wire of the heating element is a steel wire or a Kanthala wire of larger diameter tapering to a small-diameter wire at the tip.

5. The system as claimed in claim 1, wherein the anastomosis kit comprises:
an artificial tubular organ associated with the one or more artificial organs having a first end and a second end; and
a circulation unit fluidly coupled to the artificial tubular organ, and configured to enable a fluid to flow from the first end of the artificial tubular organ and to the second end of the artificial tubular organ, wherein the circulation unit comprises:
a tank configured to hold a fluid mimicking one or more of blood, lymph, and a tissue fluid; and
a pulsating pump configured to extract the fluid from the tank, the pulsating pump having varying flow rates to simulate blood flow in vessels of the living being within the artificial tubular organ.

6. The system as claimed in claim 5, wherein the anastomosis kit further comprises:
a first pressure sensor configured to measure an inlet flow rate of the fluid on the first end of the artificial tubular organ;
a second pressure sensor configured to measure an outlet flow rate of the fluid on the second end of the artificial tubular organ; and
one or more strain gauges, positioned on the artificial tubular organ, configured to measure a rate of tensile strain induced in the artificial tubular organ during the suturing process,
wherein the inlet flow rate and the outlet flow rate are compared before and after an anastomosis process performed on the artificial tubular organ to assess quality of the suturing or stapling process.

7. The system as claimed in claim 1, wherein the one or more artificial organs comprise a silicone-based material formed by mixing Part A and Part B silicone components in a 1:1 volumetric ratio,
wherein each of the one or more artificial organs comprise an artificial tissue, wherein the artificial tissue component comprises a composition of paraffin wax in a range of 70-80 % by volume, polyester synthetic cotton microfibers in a range of 15-20 % by volume, and petroleum jelly in a range of 5-10 % by volume.

8. The system as claimed in claim 1, further comprises a cooling unit configured to cool the heating element, wherein the cooling unit comprises:
a pump configured to circulate a cooling medium; and
one or more nozzles positioned at the distal end of the laparoscopic hand instrument, and configured to deliver the cooling medium to the heating element to provide a cooling to the heating element,
wherein the cooling unit is configured to provide cooling using the cooling medium selected from a group consisting of air and water, and wherein the pump is accordingly selected from a group consisting of an air pump, water pump, a vacuum pump, and a mini compressor.

9. The system as claimed in claim 7, wherein at least a portion of the one or more artificial organs include an interposed layer of biomimetic material disposed between simulated tissue layers of adjacent artificial organs,
the interposed layer being configured to undergo separation in response to application of energy selected from the group consisting of radiofrequency energy, thermal energy, and combinations thereof.

10. The system as claimed in claim 8, wherein the cooling unit comprises one or more external fans positioned near the distal end of the laparoscopic hand instrument for cooling the heating element.
